(19) United States
(12) Patent Application Publication
(10) Pub. No.: US 2024/0115399 A1
(43) Pub. Date: Apr. 11, 2024

(54) SURGICAL INSTRUMENT TO IMPLANT PRIMARY AND SECONDARY ELEMENTS

(71) Applicant: BONEARMED S.R.L, Bologna (IT)

(72) Inventor: Alessandro Russo, Bologna (IT)

(21) Appl. No.: 18/270,266

(22) PCT Filed: Dec. 23, 2021
(86) PCT No.: PCT/IB2021/062231

§ 371 Date: Jun. 29, 2023

(30) Foreign Application Priority Data
Dec. 31, 2020 (IT) 102020000032936

(51) Int. Cl. A61F 2/46

(52) U.S. Cl. CPC A61F 2/4618; A61F 2002/30787

(57) ABSTRACT

The present invention relates to a lattice support structure for one or more degenerated portions of subchondral bone of a bone epiphysis part of a human or animal joint, and to a related kit of parts and to a template assembly for the assembly thereof.

The lattice structure comprises at least one rod-shaped and substantially rectilinear rigid primary element configured to be housed within said bone epiphysis extending at least partially through said degenerated portion of subchondral bone along a respective primary extension direction; and a plurality of thread-like substantially rectilinear secondary elements configured to be housed within said bone epiphysis extending at least partially through said degenerated portion of subchondral bone along respective secondary extension directions, comprising first secondary elements configured to extend along respective first secondary extension directions and second secondary elements configured to extend along respective second secondary extension directions (Yd, Ye, Yf), the first and second secondary extension directions being oblique to one another, wherein the at least one primary element has a first transversal dimension greater than a second transversal dimension of said secondary elements.

The at least one primary element and the secondary elements are further configured to reach and cross at least partially, at respective opposite ends, cortical bone portions of said bone epiphysis.

The present invention relates to the field of implantable medical devices, and in particular to a lattice support structure for one or more degenerated portions of subchondral bone of a human or animal joint.

Arthrosis is a degenerative disease with painful symptoms caused by the progressive wear of the cartilage covering the bone joint ends. It is one of the most frequent causes of joint pain, especially in the population over 50 years of age, and most frequently affects joints subjected to greater loads such as the knee, hip and cervical and lumbar vertebrae. However, other joints such as the joints of the hands and shoulders may also be affected.

Pain in arthrosis arises primarily from the bone tissue and secondarily from the synovial membrane lining the articular surface of the joint capsule, these tissues being innervated and vascularised, unlike articular cartilage which lacks vessels and nerves. In the natural course of arthrosis, the bone tissue is depleted of its mineral and protein components, and consequently loses its mechanical strength and elasticity. The subchondral and meta-epiphyseal bone, i.e., the bone layer underneath the articular cartilage, is subjected to mechanical stresses which exceed its strength, and undergoes deformation beyond the physiological threshold, thus causing the activation of nociceptive nerve fibres and the onset of pain.

It is in fact such aggravating pain, and not the functional limitation of joint movement due to the deformity of the arthritic bone heads, which determines the need for surgery in most cases.

To date, prosthetic joint replacement is the gold standard for treating the most severe forms of arthrosis. The replacement of deformed joint surfaces with metal prosthetic components now ensures an implant survival rate which is estimated to be over 75% for hip replacements at 25 years after surgery, and over 90% for knee replacements at 20 years after surgery (Evans JT; Lancet 2019).

A first critical issue related to prosthetic surgery is the incidence of post-surgical complications. The scientific literature reports more than 15% postoperative complications, with 10% of cases requiring re-operation (David Figueroa, Arthroplasty today 2019).

A second critical point highlighted by the scientific literature is the not inconsiderable percentage of patients, up to 34% in the case of knee implants, who continue to report pain and functional limitation after the implantation of the prosthesis in the absence of other ascertainable causes. The causes of residual pain after prosthetic surgery are manifold and in some cases cannot be precisely identified.

Improving the performance of joint prostheses, particularly in the light of the second criticality highlighted above, is however hampered on the one hand by the design of the prosthesis, and on the other by the materials used.

The design of the joint prosthesis is conceived based on geometrical compromises extrapolated from anatomical and computational studies carried out on anatomical samples, and is therefore a standard design which takes into account the average of the variations of the sample under study. Regardless of the sample size, it is inevitable that for each individual the joint prosthesis represents an adaptation with respect to the population average.

In terms of materials, joint prostheses are made of metal material, typically steel or titanium alloys, and are fixed to the meta-epiphyseal bone. The fixation of metal to the meta-epiphyseal bone produces a composite which, however, has different and even opposite mechanical features to those of the natural articular surface. In fact, natural bone has a decreasing hardness gradient, as well as an increasing gradient of elasticity, going from the compact cortical bone of the diaphysis, through the metaphysis with a predominance of trabecular bone, to the cartilage covering the joint surfaces. Metal prosthetic components weigh at least an order of magnitude more with respect to the weight of the diseased cartilage and bone which is replaced by the prosthetic component. Furthermore, they have a higher hardness and lower elasticity with respect to the subchondral metaphyseal bone and articular cartilage, thereby making an anti-physiological composite material.

A more recently introduced method in orthopaedics is so-called subchondroplasty, which involves the reinforcement of degenerated subchondral bone by intraosseous injection of composite materials, e.g., tricalcium-phosphate cements. This treatment is mainly indicated for the treatment of bone oedema and not for the treatment of arthrosis. Subchondroplasty is not effective in reducing the loads on the subchondral bone and is not effective in reducing stress in the areas of subchondral bone subject to load.

Several methods and devices which can be implanted in degenerated subchondral bone have also been developed for the reduction of arthritic pain.

For example, RU 2161929 C1 discloses a surgical method which involves making a series of curved grooves along the femoral condyle in a subcortical position, at a projection of the locations of arthritic bone cysts. A plate of porous nickel-titanium with corresponding shape and size is introduced into each groove. Screws can also be inserted from the side of the joint surface to stabilise the cartilage by fixing it to the plate.

US 2011/125264 A1 discloses a series of devices and methods for the treatment of degenerated joint bone tissue.

This document envisages in particular the use of a device which can be implanted in the subchondral bone to support the areas affected by arthrosis. The device can assume various shapes depending on the area of insertion, the shape of the bone defects and/or the mode of insertion, for example it can comprise a substantially plate-like body, or a straight or curved oblong body. The insertion of the implantable device into the bone can occur by using guide wires or special surgical guide instruments.

The use of the device can be further supplemented by injections, in the area surrounding the implant, of bone cement or other carriers containing biological agents.

WO 2017/091657 A1 discloses an implantable orthopaedic device for the treatment of articular bone, comprising a first section of a generically cylindrical shape having a joint-ward end, an opposing mating end with a second section of the device, and a lateral wall extending between the two ends, said first section further comprising an axially extending central opening configured for engaging an insertion instrument.

The device comprises the aforesaid second section, having a mating end with the first section, an opposite leading end and a lateral wall extending between the mating end and the leading end. The side wall of the second section of the device comprises threads configured to facilitate penetration into the subchondral articular bone during the implantation of the device.

US 2012/185044 A1 illustrates a series of implantable devices configured to provide structural and dampening support to degenerated subchondral bone adjacent to a joint.

The device can be made in the form of a shaped plate, possibly provided with holes. In other designs, the device can comprise an elongated base plate and a series of tapered strut elements extending transversally from both faces of the base plate. In still other forms, the system can consist of a series of separate strut elements of varying geometry (e.g., plate-like or shaped like tubular or circular elements), configured for modular insertion into the subchondral bone.

The disclosed devices have one or more holes or slots to accommodate guide pins to facilitate the insertion of the device within the subchondral bone.

US 2013/035561 A1 also discloses methods and devices for the treatment of arthritic joint pain. Among the various treatments described, the document also envisages the mechanical strengthening of the subchondral region of the bone affected by osteoarthritis by means of a reinforcing member to be positioned in situ. The element can be implanted without constraints with the bone or can be fixed thereto. The document generically discloses that the reinforcing member can have various shapes, for example it can be made as a substantially planar or elongated member.

The various documents of the prior art discussed above teach how to support arthritic portions of subchondral bone by inserting variously shaped elements near the bone defects, so as to assume part of the loads on the latter.

However, the Applicant has observed that none of the devices disclosed by the above-mentioned known technical documents is capable of operating an effective “global” redistribution of stresses, such as to transfer part of the loads acting on diseased bone regions to healthy bone regions. Since the disclosed devices only operate locally, distributing loads in regions close to the defects and therefore consisting almost entirely of compromised bone tissue, they are not capable of substantially decreasing the deformation suffered by the diseased bone, nor consequently reducing the pain felt by the patient. However, it is observed that if the bone defects spread to the surrounding areas, it would be necessary to replace such inserted devices with larger and/or differently shaped ones.

However, the Applicant has noted that the implantation of devices with a more complex shape and larger dimensions, such as those with a plate-like shape, cylindrical, or comprising protruding strut elements, requires particularly invasive surgical insertion procedures with the removal of non-negligible portions of the patient's native bone, in order to create the niches for housing the devices. However, in these cases the surgical procedure of preparing the bone and inserting the support elements is also complex and difficult to reproduce.

The Applicant has therefore perceived that it would be advantageous to be able to redistribute the loads on degenerated subchondral bone portions also over larger bone sections with respect to what is made possible by the devices of the prior art, and in particular by transferring part of these loads also to healthy bone regions, without however increasing—or even decreasing—the invasiveness of the implantable device.

The technical problem underlying the present invention is therefore that of effectively relieving the loads acting on portions of degenerated subchondral bone through the use of a minimally invasive device which can be implanted by means of a simple procedure by medical personnel.

Therefore, in a first aspect, the present invention relates to a lattice support structure for one or more degenerated portions of subchondral bone of a bone epiphysis part of a human or animal joint, comprising:
at least one rod-shaped and substantially rectilinear rigid primary element, configured to be housed within said bone epiphysis extending at least partially through said degenerated portion of subchondral bone along a respective primary extension direction, and
a plurality of substantially rectilinear thread-like secondary elements configured to be housed within said bone epiphysis extending at least partially through said degenerated portion of subchondral bone along respective secondary extension directions, comprising first secondary elements configured to extend along respective first secondary extension directions, and second secondary elements configured to extend along respective second secondary extension directions, the first and second secondary extension directions being oblique to one another,
in which said at least one primary element has a first transversal dimension greater than a second transversal dimension of said secondary elements.

Advantageously, the at least one primary element and the secondary elements are configured to reach and cross at least partially, at respective opposite ends, cortical bone portions of said bone epiphysis.

In the present description and the appended claims, “degenerated bone portion”, sometimes hereinafter referred to as “degenerated portion of bone”, or similar expressions, means diseased bone tissue which has lost its physiological mechanical and elastic properties due to arthrosis or analogous degenerative diseases.

In the present description and the appended claims, “cortical bone portion”, “cortical bone” and similar expressions mean a bone layer comprising mainly compact lamellar bone tissue and forming an outer cortex of the articular bone epiphysis for which the lattice structure of the invention is intended.

In the present description and the appended claims, the term “oblique” means a direction which is neither parallel to nor coincident with a second direction.

In the present description and the appended claims, the terms “proximal” and “distal” are used to indicate a point closer to, respectively farther away from, the bone epiphysis in which the lattice structure of the invention is assembled.

In the present description and the appended claims, the expressions “transversal”, “transversally” and the like, used with reference to bodies or elements of elongated shape, indicate a direction substantially orthogonal with respect to a reference direction along which the body or element in question primarily extends.

In the present description and the appended claims, the expressions “above”, “below”, “top”, “bottom” refer to mutual positions of elements of the lattice structure oriented as in perspective views (e.g., FIG. 1, 5) in frontal views (e.g., FIG. 3, 6) or side views (e.g. FIG. 4).

In the present description and the appended claims, “development direction of the main body of the template” means a direction, understood as a curved line and/or including a plurality of rectilinear segments, which goes through the main body of the template from one free end to the other.

In the present description and the appended claims, “development plane of the main body of the template” means a plane containing the development direction of the main body of the template, such plane substantially coinciding with a horizontal plane in the perspective views of FIGS. 10 and 11.

In a second aspect thereof, the present invention relates to a lattice support structure for one or more degenerated portions of subchondral bone of a bone epiphysis part of a human or animal joint, comprising:
at least one rod-shaped and substantially rectilinear rigid primary element, housed within said bone epiphysis extending at least partially through said degenerated portion of subchondral bone along a respective primary extension direction, and
a plurality of substantially rectilinear thread-like secondary elements housed within said bone epiphysis extending at least partially through said degenerated portion of subchondral bone along respective secondary extension directions, comprising first secondary elements extending along respective first secondary extension directions, and second secondary elements extending along respective second secondary extension directions, the first and second secondary extension directions being oblique to one another,
in which said at least one primary element has a first transversal dimension greater with respect to a second transversal dimension of said secondary elements, and
in which the at least one primary element and the secondary elements reach and cross at least partially, at their respective opposite ends, cortical bone portions of said bone epiphysis.

The Applicant has surprisingly found that the use of at least one rigid primary element, in cooperation with a series of substantially thread-like secondary elements, to form a lattice structure as defined above in relation to the first two aspects of the invention, exerts an advantageous effect of lightening the loads acting on a degenerated portion of subchondral bone.

At least one rigid primary element, shaped like a rectilinear rod and having a larger transversal dimension with respect to the secondary elements, gives mechanical stability to the structure and helps support the loads of the degenerated bone portion surrounding it.

The thread-like secondary elements, extending in the bone portion along oblique directions, form a real lattice substructure with particularly strong supporting “mesh”, capable of elastically absorbing and effectively redistributing the stresses due to the loads acting on the joint over the whole volume of subchondral and meta-epiphyseal bone of the bone epiphysis in which the lattice structure is implanted.

Advantageously, since both the primary and secondary elements extend up to intercepting the cortical portions of the bone epiphysis at both ends, the lattice structure thus formed rests on such cortical portions, which are much denser, more rigid and therefore stronger with respect to the trabecular bone of which the subchondral bone region of the joint consists.

This cortical abutment allows the loads to bypass the degenerated trabecular bone in the meta-epiphyseal subchondral region, unload onto the compact healthy cortical bone and redistribute to the healthy subchondral bone, thus providing optimal structural support.

In fact, the Applicant has experimentally verified that the lattice structure of the invention allows to reduce the load peaks acting on degenerated articular bone portions by more than 30%. The decrease in load implemented by the structure according to the invention is capable of decreasing the deformations of the degenerated bone portions by more than 20%, bringing the deformation levels to which the degenerated subchondral bone is subjected below the physiological threshold. Reducing the loads and deformities leads to a significant reduction (or even disappearance) of the pain felt by the patient when the joint is loaded again, for example, if the joint concerned is the knee or hip, when the patient stands up from a sitting or lying position, or when walking.

Moreover, the lattice structure of the invention is particularly light, has a minimal footprint and consequently its assembly does not require the removal of bone portions of significant size, with consequent notable advantages from the point of view of minimum invasiveness, preservation of natural tissue and post-operative recovery of the patient.

In a third aspect, the invention relates to a kit of parts for assembling a lattice support structure for one or more degenerated portions of subchondral bone of a bone epiphysis part of a human or animal joint, said kit comprising:
at least one rod-shaped and substantially rectilinear rigid primary element;
a plurality of substantially rectilinear thread-like secondary elements, comprising first secondary elements and second secondary elements; and
a surgical template comprising an elongated main body and at least one pin bearing a fixing member for an end of said at least one primary element, said at least one pin being configured to extend in use along a primary extension direction of said at least one primary element within said degenerated portion of subchondral bone, and said main body comprising first secondary holes defining respective first secondary extension directions for said first secondary elements, and second secondary holes defining respective second secondary extension directions for said second secondary elements, the first and second secondary extension directions being oblique to one another;
in which said at least one primary element has a first transversal dimension greater than a second transversal dimension of said secondary elements, and
in which the at least one primary element and the secondary elements are configured to reach and cross at least partially, at both the respective ends, cortical bone portions of said bone epiphysis.

The kit of parts in accordance with the third aspect of the invention enables simple and precise assembly of the lattice support structure, the advantages of which have been outlined above in relation to the first two aspects of the invention.

In a fourth aspect, the invention relates to a template assembly including a surgical template comprising an elongated main body comprising at least one fixing member configured to cooperate with at least one corresponding fixing seat of at least one rod-shaped and substantially rectilinear rigid primary element.

The main body of the template comprises first and second secondary through holes configured to respectively house first secondary elements and second secondary elements which are thread-like and substantially rectilinear, in which the first secondary holes define respective first secondary extension directions for the first secondary elements, and the second secondary holes define respective second secondary extension directions for the second secondary elements, the first and second secondary extension directions being oblique to one another.

The provision of a surgical template and a template assembly, and as outlined in relation to the third and fourth aspects of the invention, allows to define in a simple and rapid manner the secondary extension directions along which the secondary elements are to be inserted, starting from the reference consisting of the extension direction of the primary element, to the end of which the template is fixed, so as to allow the assembly of a well-defined and geometrically precise lattice structure.

The present disclosure further relates to a method for assembling a lattice support structure for a degenerated portion of subchondral bone of a bone epiphysis part of a human or animal joint, comprising the steps of:
arranging at least one rod-shaped, substantially rectilinear rigid primary element;
arranging a plurality of substantially rectilinear thread-like secondary elements, comprising first secondary elements and second secondary elements;
in which said at least one primary element has a first transversal dimension greater than a second transversal dimension of said secondary elements;
arranging a template assembly according to the fourth aspect of the invention set out above;
forming, by means of bone coring, at least one housing seat through said degenerated bone portion for the at least one primary element, said housing seat crossing the bone epiphysis from side to side and comprising accesses facing opposite cortical bone portions of said bone epiphysis;
inserting the at least one primary element into said at least one housing seat;
constraining the template assembly to said at least one primary element by fixing the at least one fixing member of the template at the at least one corresponding fixing seat of said at least one primary element, accessing said at least one fixing seat through one of the accesses of the housing seat;
inserting the secondary elements into the degenerated portion of subchondral bone using a drilling tool, sliding the secondary elements through the secondary holes of the template; and
removing the template assembly by releasing the at least one fixing member from the corresponding at least one fixing seat of the at least one primary element.

The present disclosure further relates to a method for assembling a lattice support structure for a degenerated portion of subchondral bone of a bone epiphysis part of a human or animal joint, comprising the steps of:
arranging at least one rod-shaped, substantially rectilinear rigid primary element;
arranging a plurality of substantially rectilinear thread-like secondary elements, comprising first secondary elements and second secondary elements;
in which said at least one primary element has a first transversal dimension greater than a second transversal dimension of said secondary elements;
arranging a surgical template comprising an elongated main body and said at least one pin having a fixing member for one end of said at least one primary element, said at least one pin being configured to extend in use along a primary extension direction of said at least one primary element within said degenerated portion of subchondral bone, and said main body comprising first secondary holes defining respective first secondary extension directions for said first secondary elements, and second secondary holes defining respective second secondary extension directions for said second secondary elements, the first and second secondary extension directions being oblique to one another;
forming, by means of bone coring, at least one housing seat through said degenerated bone portion for the at least one primary element, said housing seat crossing the bone epiphysis from side to side and comprising accesses facing opposite cortical bone portions of said bone epiphysis;
inserting the at least one primary element into said at least one housing seat;
constraining the template to said at least one primary element by fixing the fixing member of the pin of the template at the fixing seat borne by the end of the at least one primary element, accessing said end through one of the accesses of the housing seat;
inserting the secondary elements into the degenerated portion of subchondral bone using a drilling tool, passing the secondary elements through the secondary holes of the template; and
removing the template by releasing the pin from the end of the at least one primary element.

The aforesaid methods allow to obtain a lattice support structure for one or more degenerated portions of subchondral bone, having all the advantages outlined above in relation to the first aspect of the invention.

In particular, such methods allow to assemble the lattice support structure in a simple manner and with a high degree of precision in the positioning of the main and secondary elements, by means of alignment with respect to the pin or the respective holes borne by the template, which define the respective extension directions of each element.

Furthermore, the disclosed methods are minimally invasive to the patient as they require the removal of a minimal portion of degenerated bone tissue, essentially corresponding to the bone core extracted to form the housing seat for the primary element, and the minimal bone portion which is ejected during drilling to insert the secondary elements.

The present invention can be presented in one or more of its aspects or one or more of the preferred features reported below, which can be combined with one another as preferred according to the application requirements.

Preferably, the first secondary extension directions of the first secondary elements lie in a first plane.

Preferably, the second secondary extension directions of the second secondary elements lie in a second plane.

Preferably, moreover, the first plane and the second plane are substantially parallel to one another.

Preferably, the first secondary extension directions of the first secondary elements are substantially parallel to one another.

Preferably, the second secondary extension directions of the second secondary elements are substantially parallel to one another.

Preferably, the first secondary elements and the second secondary elements are substantially side by side or in contact at the first crossing points between the first secondary extension directions and the second secondary extension directions.

Preferably, the first secondary extension directions of the first secondary elements are substantially orthogonal to the second secondary extension directions of the second secondary elements.

This configuration of the secondary elements is the optimal one from the point of view of load redistribution and the easiest to implement during the assembly of the lattice structure.

Preferably, the main body of the template comprises a central portion extending between two end portions or free ends.

Preferably, said end portions or free ends extend from the same side of the template with respect to said central portion.

Preferably, the main body of the template is shaped to at least partially surround a bone epiphysis.

Preferably, the main body of the template is substantially C-shaped.

Preferably, a development direction of the main body of the template is contained in a development plane.

Preferably, the main body of the template is substantially planar, neglecting a thickness thereof in the direction transversal to said development plane.

Preferably, said first plane is substantially parallel to said development plane of the main body of the template.

Preferably, said second plane is substantially parallel to said development plane of the main body of the template.

Preferably, said third plane is substantially parallel to said development plane of the main body of the template.

Preferably, said fourth plane is substantially parallel to said development plane of the main body of the template.

Preferably, said primary extension direction along which the at least one pin of the template extends is parallel to said development plane of the main body of the template.

Preferably, the main body of the template comprises a first rectilinear portion and a second rectilinear portion substantially extending orthogonal to the first rectilinear portion.

In embodiments, the first rectilinear portion is defined at the central portion of the main body of the template.

In embodiments, the second rectilinear portion is defined at one of the free ends of the main body of the template.

In embodiments, the first rectilinear portion and the second rectilinear portion are respectively defined at the free ends of the main body of the template.

In embodiments, the main body of the template comprises a third rectilinear portion extending substantially orthogonal to the first rectilinear portion and substantially parallel to the second rectilinear portion.

Preferably, the first secondary holes are borne by the first rectilinear portion of the template, and the second secondary holes are borne by the second rectilinear portion of the template.

Preferably, moreover, the first and second secondary holes extend transversally with respect to said first, respectively second, rectilinear portion of the template.

In embodiments, said at least one pin is borne by the first rectilinear portion of the main body of the template.

Preferably, said at least one pin of the template extends substantially parallel to said first secondary holes.

In embodiments, the plurality of secondary elements further comprises third secondary elements configured to extend along respective third secondary extension directions which are oblique with respect to the first secondary extension directions and/or the second secondary extension directions.

The presence of this third group of secondary elements with a third orientation with respect to the first two groups further strengthens the lattice structure and allows the degenerated portion of subchondral bone to be unloaded by means of an even more effective redistribution of the stresses acting thereon.

Preferably, said third secondary extension directions lie in a third plane substantially parallel to said first plane and/or said second plane in which said first and second extension directions lie, respectively.

Preferably, said third extension directions are oriented at about 45° with respect to the first secondary extension directions and/or the second secondary extension directions.

This configuration is particularly advantageous when the first secondary extension directions and the second secondary extension directions are orthogonal to one another.

When the third secondary elements are present, the plurality of secondary elements preferably also comprises fourth secondary elements configured to extend along respective fourth secondary extension directions which are oblique with respect to the third secondary extension directions as well as the first secondary extension directions and/or the second secondary extension directions.

Preferably, said fourth secondary extension directions lie in a fourth plane substantially parallel to said third plane in which said third secondary extension directions lie, as well as to said first plane and/or said second plane in which said first and second secondary extension directions lie, respectively.

Preferably, the third secondary elements and the fourth secondary elements are substantially side by side or in contact at the second crossing points between the third secondary directions and the fourth secondary directions.

The provision of third and fourth secondary elements extending in respective third and fourth secondary extension directions which are oblique to one another, allows the formation of a second ‘mesh’ substructure, distinct from the first substructure formed by the first and second secondary elements. This allows the stresses to be redistributed in parallel on two distinct planar regions of the bone epiphysis.

Preferably, said fourth secondary extension directions are orthogonal with respect to the third secondary extension directions.

Furthermore, said fourth extension directions are preferably oriented at about 45° with respect to the first secondary extension directions and/or the second secondary extension directions.

In this case, preferably the main body of the template comprises third secondary holes defining respective third secondary extension directions for said third secondary elements, and fourth secondary holes defining respective fourth secondary extension directions for said fourth secondary elements, the third and fourth secondary extension directions being oblique to one another.

In embodiments, the third secondary holes are borne by the first rectilinear portion of the template, and the fourth secondary holes are borne by the second rectilinear portion of the template, extending substantially orthogonal to the first rectilinear portion of the template.

Preferably, the third secondary holes and the fourth secondary holes extend transversally to said first, respectively second, rectilinear portion of the template.

Preferably, in this case the at least one pin of the template extends at about 45° with respect to said third secondary holes and said fourth secondary holes of the template.

In embodiments, the main body of the template includes a connecting portion extending between the first rectilinear portion and the second rectilinear portion.

Preferably, said connecting portion is oriented at about 45° with respect to the first and second rectilinear portions of the template.

In embodiments, the at least one pin is borne by said connecting portion of the main body of the template.

Preferably, the primary extension direction of the at least one primary element is substantially orthogonal to said second secondary extension directions of the first secondary elements.

Preferably, the first secondary directions coincide with the axes of the first secondary holes.

Preferably, the second secondary directions coincide with the axes of the second secondary holes.

Preferably, the third secondary directions coincide with the axes of the third secondary holes.

Preferably, the fourth secondary directions coincide with the axes of the fourth secondary holes.

Preferably, the template comprises a first fixing member, configured to cooperate with a corresponding first fixing seat of a rod-shaped and substantially rectilinear rigid first primary element, and a second fixing member, configured to cooperate with a corresponding second fixing seat of a rod-shaped and substantially rectilinear rigid second primary element.

Preferably, the template comprises a first pin bearing said first fixing member at a proximal end thereof.

Preferably, said first pin is substantially parallel to said development plane of the main body of the template.

Preferably, the template comprises a second pin bearing said second fixing member at a proximal end thereof.

Preferably, said second pin is substantially parallel to said development plane of the main body of the template and substantially parallel to said first pin.

In embodiments, the template assembly according to the fourth aspect of the invention further comprises said first thread-like and substantially rectilinear secondary elements inserted into the first secondary holes.

In embodiments, the template assembly according to the fourth aspect of the invention further comprises second thread-like and substantially rectilinear secondary elements inserted into the second secondary holes.

In embodiments, the template assembly according to the fourth aspect of the invention further comprises third thread-like and substantially rectilinear secondary elements inserted into the third secondary holes.

In embodiments, the template assembly according to the fourth aspect of the invention further comprises fourth thread-like and substantially rectilinear secondary elements inserted into the fourth secondary holes.

The pre-assembled configuration of the template assembly, in which the template assembly is supplied to the medical staff with the secondary elements already inserted into the respective secondary holes of the template, is particularly advantageous as it saves time during the surgical procedure and avoids potential errors in the mutual positioning and mounting of the secondary elements on the template.

Preferably, the at least one primary element has a substantially circular cross section, the first transversal dimension of the at least one primary element being in such a case a first diameter.

Preferably, moreover, the secondary elements have a substantially circular cross section, the second transversal dimension of the secondary elements being in such a case a second diameter.

By way of example, the secondary elements are Kirschner wires.

Preferably, the first diameter of the at least one primary element is between 3 and 10 mm, more preferably between 4 and 8 mm.

Preferably, the second diameter of the secondary elements is between 0.3 and 2 mm, more preferably between 0.8 and 1.6 mm.

Preferably, a ratio of the first diameter of the at least one primary element to the second diameter of the secondary elements is between 1.5 and 34, more preferably between 2.5 and 10.

Preferably, the secondary elements are threaded at least at one respective insertion end in the bone.

Alternatively or additionally, the secondary elements are also threaded at a respective end opposite the insertion end in the bone.

The screw thread allows a real anchorage to the cortical bone portion, helping to stabilise the position of the secondary elements within the lattice structure.

In preferred embodiments, the at least one primary element comprises a tubular body.

This shape of the primary element allows the corresponding housing seat to be made in the subchondral bone in a simple manner for the medical staff, by means of bone coring operations.

Preferably, in this case, said first diameter of the at least one primary element corresponds to an external diameter of said tubular body.

Preferably, the tubular body of the at least one primary element bears a plurality of through openings.

The presence of openings in the tubular body allows the primary element to interact with the secondary elements, as will become clear from the following description, conferring greater stability to the lattice structure. Furthermore, the presence of such openings stimulates the bone to regrow through the openings and into the internal lumen of the primary element, promoting the osseointegration thereof and the filling of the removed bone volume.

In this case, preferably the secondary extension directions of the secondary elements intersect the primary extension direction of the at least one primary element. Therefore, at least some of the secondary elements are preferably configured to extend through corresponding through openings of the tubular body of the at least one primary element.

In this case, the primary and secondary elements are interconnected with one another, making the lattice structure more robust and stable.

In some embodiments, the at least one primary element includes a first primary element configured to extend along a first primary extension direction, and a second primary element configured to extend along a second primary extension direction.

The addition of further primary elements provides more structural support to the degenerated subchondral bone, allows the structure to be strengthened, providing further possible interconnection points for the secondary elements.

More preferably, the first primary direction and the second primary direction are substantially orthogonal to one another.

Preferably, the first primary extension direction of the first primary element intersects the second primary extension direction of the second primary element.

In such a case, the second primary element is preferably configured to extend through corresponding through openings of the tubular body of the first primary element.

In this case, the second primary element has a transversal section having a third diameter which is smaller with respect to the first diameter of the first primary element, and in particular compatible with the size of the openings of the first primary element in which it is inserted.

In this configuration, the openings of the first primary element are advantageously used to connect the second primary element, forming an even more robust structural assembly of primary elements and capable of conferring an even greater support to the degenerated subchondral bone.

Preferably, the at least one primary element comprises, at one end thereof, a fixing seat for the surgical template described above with reference to the second aspect of the invention.

Said fixing seat is preferably adapted to cooperate with a corresponding fixing member of the template.

More preferably, the fixing seat of the at least one primary element comprises a threaded hole, and the fixing member of the template comprises a corresponding threaded member.

In some embodiments, anchoring elements configured to anchor the secondary elements at one or both ends to the cortical bone portion can also be envisaged.

For example, such anchoring elements can comprise locking bands arranged around the ends of the secondary elements emerging from the cortical bone portion.

In this case, the assembly method further comprises a step of anchoring one or both ends of each secondary element to said cortical bone portion by applying said anchoring elements.

The at least one primary element, when configured as a tubular element, can advantageously be used as a carrier of substances or materials to promote bone regrowth in situ.

For example, in preferred embodiments, the internal lumen of the at least one primary element houses therein a filler which in preferred embodiments is an autologous bone graft of the patient for whom the lattice structure is intended.

In this case, the tissue inserted in the primary element can be the same tissue which is first removed, by bone coring, from the degenerated bone portion to create the housing seat for the primary element.

Alternatively, the filler can be a homologous bone graft or a synthetic bone graft.

In all the cases described above, the filler can be supplemented with the patient's own stem cells and/or growth factors to promote bone regrowth.

Preferably, the at least one primary element and/or the secondary elements are made of one or more materials selected from titanium, steel, tantalum, ceramic materials, polymer materials, including for example composite polymer materials, ceramic materials, including for example composite ceramic materials, carbon fibre and graphene.

In some embodiments, the at least one primary element and/or the secondary elements are configured to transfer regenerative stimuli to the degenerated portion of subchondral bone when subjected to one or more conditions selected from an electric current, a static and/or pulsed magnetic field, ultrasound and hyperthermia.

These practices can further contribute to reducing the patient's perception of nociceptive nerve stimuli.

Preferably, said bone epiphysis is selected from a bone epiphysis of a knee joint, a hip joint, a shoulder joint, an ankle joint, a foot joint, and a vertebral body, more preferably from a bone epiphysis of a knee joint and a hip joint, more preferably being part of a knee joint.

When the joint concerned is the knee, the bone epiphysis within which the lattice structure is assembled is preferably selected from the tibial plateau, the distal femur and the patella, more preferably being the tibial plateau.

Preferably, the first and second planes in which the aforesaid first and second secondary extension directions of the first and second secondary elements of the lattice structure respectively lie are substantially orthogonal to a longitudinal axis of the bone of which the bone epiphysis is a part.

When the affected joint is the knee and the bone epiphysis is the tibial plateau, the first and second planes are substantially transversal to the bone epiphysis.

Preferably, the components of the lattice structure assembly kit according to the invention are patient-specific.

Therefore, the invention preferably envisages the ad hoc definition of the configuration of the lattice structure, in terms of the number of dimensions and components of the assembly kit, and in terms of the positioning of the components during the assembly method.

Alternatively, it is possible to make a kit of parts comprising the various components in a standardised number and size.

Preferably, the disclosed assembly methods comprise a preliminary step of identifying the degenerated subchondral bone portion(s) within the bone epiphysis.

Preferably, said step of identifying the degenerated bone portion comprises performing an analysis of the loads acting on the subchondral bone in a known load situation.

For example, in the case in which the bone epiphysis is the tibial plateau of a knee joint, said known load situation can be the distribution of loads acting on the tibial plateau during a steady-step gait of a subject of known weight.

Preferably, the step of identifying the degenerated bone portion further comprises defining a three-dimensional model, e.g., a CAD model, of the loads acting in the known load situation from diagnostic images of the patient, e.g., CT or magnetic resonance imaging.

Preferably, the step of identifying the degenerated bone portion further comprises analysing said three-dimensional model by means of finite element analysis (FEM).

Preferably, the disclosed assembly methods further comprise a step of modelling the components of the lattice structure based on the evidence of the FEM analysis.

Further features and advantages of the present invention will be more evident from the following detailed description of certain preferred embodiments thereof made with reference to the appended drawings. The different features in the individual configurations can be combined together as desired.

### IN THE APPENDED DRAWINGS

FIG. 1 is a schematic perspective view of a lattice support structure according to a first embodiment of the invention, assembled at a bone epiphysis;

FIG. 2 is a schematic top view of the lattice structure of FIG. 1;

FIG. 3 is a schematic front view of the lattice structure of FIG. 1;

FIG. 4 is a schematic side view of the lattice structure of FIG. 1;

FIG. 5 is a schematic perspective view of a lattice structure according to a second embodiment of the invention, assembled at a bone epiphysis;

FIG. 6 is a schematic front view of the lattice structure of FIG. 5;

FIG. 7 is a schematic perspective view of a lattice support structure according to a third embodiment of the invention, assembled at a bone epiphysis;

FIG. 8-11 schematically illustrate some components of a kit for assembling a lattice support structure according to the invention;

FIG. 12-14 illustrate steps of a method for assembling a lattice support structure according to the invention;

FIG. 15-16 schematically illustrate bone epiphysis models used in experimental tests related to the lattice structure of the invention; and

FIG. 17-18 schematically illustrate some components of a kit for assembling a lattice support structure according to the invention.

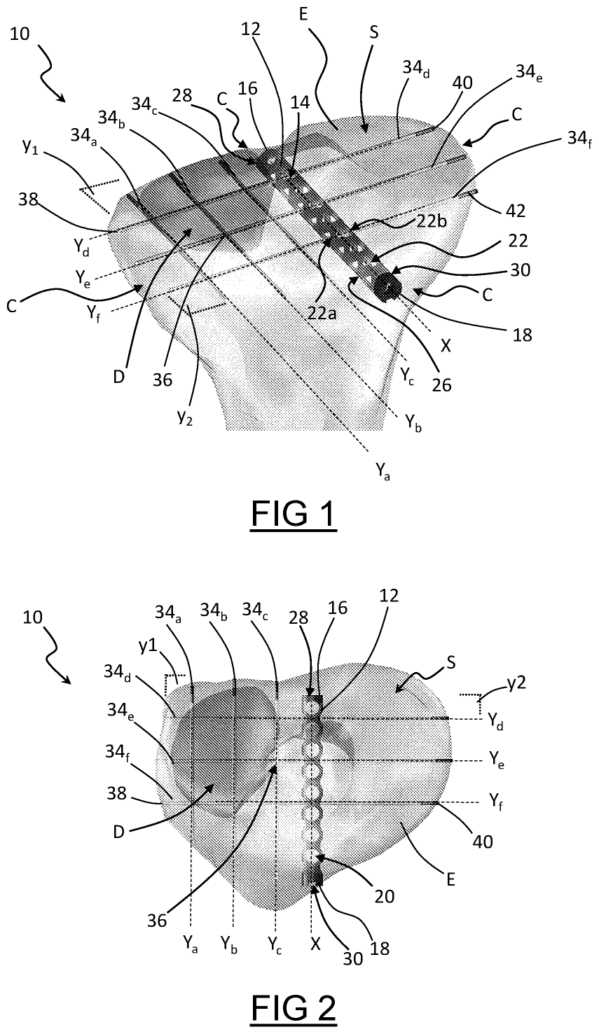

With reference to FIG. 1-4, a lattice support structure 10 for one or more degenerated portions of subchondral bone of a bone epiphysis part of a human or animal joint is now described.

The lattice structure 10 is in particular assembled at a bone epiphysis E, sometimes epiphysis E for short, which in the example shown is a tibial plate of a knee joint.

The bone epiphysis E comprises one or more degenerated portions of subchondral bone, characterised by the presence of bone with deteriorated mechanical features, for example due to arthritic phenomena, and therefore rendered unsuitable for sustaining loads.

In the various figures, a degenerated portion D of subchondral bone is schematically shown (sometimes referred to as degenerated bone portion D for brevity). The degenerated bone portion D is shown for the sake of convenience only on the joint surface S of the bone epiphysis E, but includes portions of subchondral bone located up to more than one centimetre below the joint surface S.

The lattice structure 10 comprises a rod-shaped rectilinear rigid primary element 12, extending partly within said degenerated bone portion 12 along a primary extension direction X.

In the example shown, the primary extension direction X is substantially orthogonal to a longitudinal axis A (shown in FIG. 4) of the bone of which the bone epiphysis E is a part.

For example, if the bone epiphysis E is a tibial plate as in the case shown in the various figures, the primary extension direction X lies in a transversal plane with respect to the bone epiphysis E. In particular, in the illustrated lattice structure 10, the primary extension direction X corresponds to an antero-posterior direction, as better seen in the side view of FIG. 4.

The primary element 12, visible enlarged in the representation of FIG. 8, preferably comprises a tubular body 14 extending between ends 16, 18 and defining an internal lumen 20. The tubular body 14 preferably has a circular cross-section with a first external diameter d₁.

In the preferred embodiment illustrated, the primary element 12 further comprises a plurality of through openings 22 arranged throughout the tubular body 14. The openings 22 are preferably arranged, as illustrated, along rectilinear rows 24 extending parallel to the extension direction X of the primary element 12, and two by two located in diametrically opposite positions of the tubular body 14.

The openings 22 belonging to diametrically opposite rows 24 are also preferably aligned in pairs along a respective orthogonal direction T1, T2 to the primary extension direction X, see for example the pair of openings 22a, 22b, aligned in the direction T1, and the pair of openings 22c, 22d, aligned in the direction T2, shown in FIG. 8.

The primary element 12 is inserted into a corresponding housing seat 26 made by bone coring in the bone epiphysis E at the degenerated portion D of subchondral bone. The housing seat 26 for the primary element 12, shown in FIG. 1, is in particular a through seat, such that it crosses the bone epiphysis E from side to side along the extension direction X of the primary element 12. The housing seat 26 thus comprises accesses 28, 30 facing cortical portions C located in opposite regions of the bone epiphysis E, with respect to a median plane of the epiphysis E.

As will be explained below, the specific location of the primary element 12 in the bone epiphysis E, and in particular the orientation of the relative extension direction X, are chosen following a patient-specific assessment, which is followed by the design of the lattice structure 12.

The length of the primary element 12 is previously chosen according to the position of the housing seat 26 within the bone epiphysis E, so that both ends 16, 18 of the primary element 14 reach and cross opposite cortical portions C of the epiphysis E. In this manner and as illustrated, the ends 16, 18 of the primary element 12 rest on cortical bone tissue surrounding the accesses 28, 30 of the housing seat 26, from which said ends 16, 18 partially emerge. It should be noted that in FIG. 4 the end 16 of the primary element 12 is partially covered by protruding structures of the epiphysis E, but it emerges, as does the end 18, from the access 28 of the housing seat 26.

This cortical abutment of the primary element 12 allows it to bypass the depleted trabecular bone tissue of the damaged bone portion D, transferring the loads involved to healthy subchondral bone tissue and especially to the healthy cortical portions C.

At the end 18 thereof, the primary element 12 further comprises a fixing seat 32 (shown in FIG. 8) for a surgical template 52, 152 used for the assembly of the lattice structure 10. In particular, the fixing seat 32 includes a screw thread (not illustrated) made on the internal wall of the tubular body 14, adapted to cooperate with a corresponding fixing member 70, 170 of the template 52, 152, as will be detailed below.

The lattice structure 10 according to the first embodiment of the invention, illustrated in FIG. 1-4, further comprises a plurality of thread-like secondary elements 34.

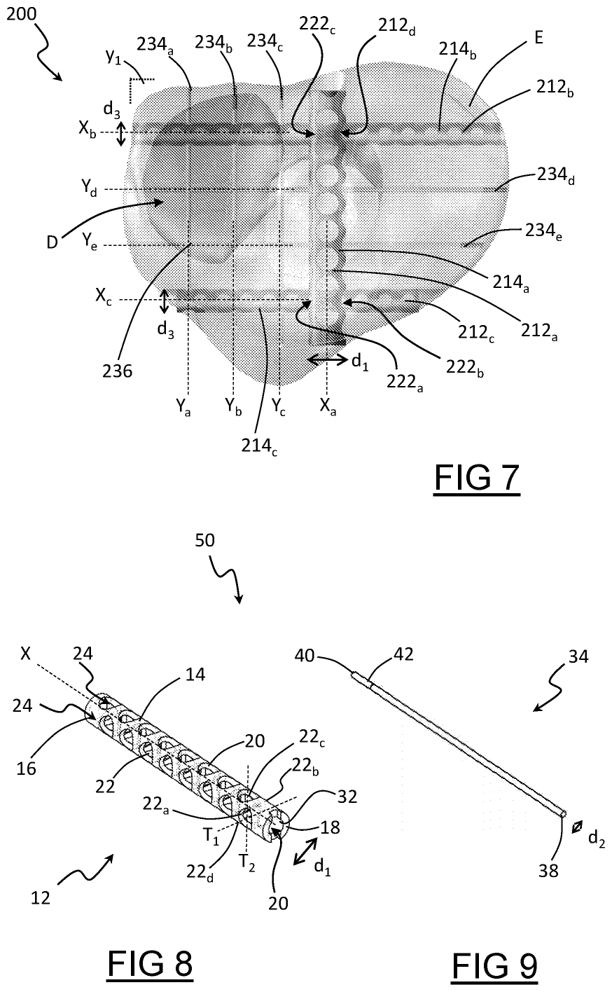

The secondary elements 34, as further illustrated in FIG. 7, have a circular cross-section having a second diameter d₂. The secondary elements 34 are elastic elements capable of undergoing a certain degree of bending.

The second diameter d₂ of the secondary elements 34 is smaller with respect to the first diameter d₁ of the primary element 12, the ratio between the first diameter d₁ of the primary element and the second diameter d₂ of the secondary elements 34 preferably being between 1.5 and 34, more preferably between 2.5 and 10.

The secondary elements 34 are distributed in mutual positions such as to create an elastic lattice substructure to support and redistribute the loads.

In fact, first secondary elements 34a, 34b, 34c are included, three in the example illustrated in FIG. 1-4, extending along respective first secondary directions Ya, Yb, Yc, and second secondary elements 34d, 34e, 34f, also three by way of example, extending along respective second secondary directions Yd, Ye, Yf. For the sake of simplicity, reference will sometimes be made in the following to the secondary extension directions as a whole with the reference Y.

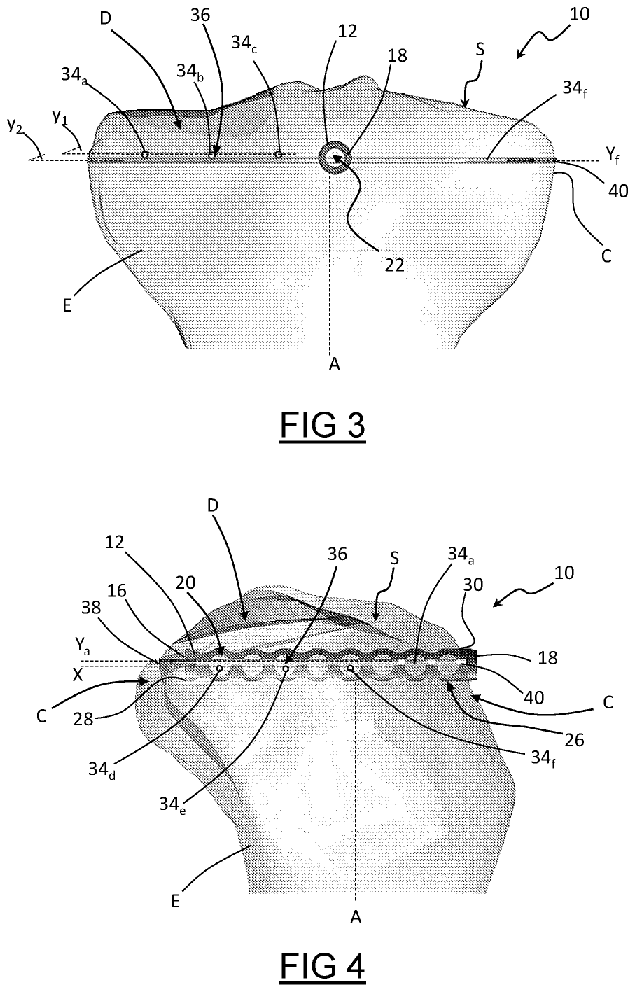

The first secondary directions Ya, Yb, Yc extend in a same first plane y1, preferably orthogonal to the longitudinal axis A (shown in FIG. 3-4) of the bone of which the epiphysis E is a part. In the case shown, the first plane y1 is a plane substantially transversal with respect to the bone epiphysis E.

Similarly, the second secondary directions Yd, Ye, Yf extend in a same second plane y2, which is also preferably orthogonal to the longitudinal axis A of the epiphysis E, and substantially parallel to the first plane y1.

As clearly shown in FIG. 2, the first secondary directions Ya, Yb, Yc are further parallel to one another, and the second secondary directions Yd, Ye, Yf are parallel to one another.

In the embodiment of FIG. 1, the second plane y2 extends below the first plane y1, slightly farther away from the joint surface S (see for example FIG. 3), the first secondary elements 34a, 34b, 34c thus run closer to the joint surface S with respect to the second secondary elements 34d, 34e, 34f. It is of course possible to envisage an inverted arrangement in which the second plane y2 extends closer to the joint surface S with respect to the first plane y1.

Advantageously, the first secondary directions Ya, Yb, Yc and the second secondary directions Yd, Ye, Yf are oblique to one another. In other words, the first secondary elements 34a, 34b, 34c and the second secondary elements 34d, 34e, 34f intersect at a series of intersection points 36 (only one indicated for simplicity in FIG. 1-4).

As better seen in FIG. 3, the first and second planes y1 and y2 are close to one another, so that the first secondary elements 34a, 34b, 34c and the second secondary elements 34d, 34e, 34f are substantially side by side or in some cases in contact at the intersection points 36.

As shown in FIG. 1, the configuration of the secondary elements 34 in terms of number and arrangement is determined so as to create a thickening of the intersection points 36 at the degenerated bone portion D. Thereby, greater elastic support is created in the most weakened bone region.

In particular, as can be seen in FIG. 2, the first secondary directions Ya, Yb, Yc and the second secondary directions Yd, Ye, Yf are orthogonal to one another.

As with the primary element 12, the length of the secondary elements 34 is also determined based on their positioning within the bone epiphysis E so that both ends 38, 40 of each secondary element 34 reach and cross opposite cortical portions C with respect to a median plane of the bone epiphysis E, partially emerging from such opposite cortical portions C of the epiphysis E.

In particular, it is possible to establish in advance the correct length of the secondary elements 34 based on the bone section into which they are to be inserted, or alternatively, once the insertion end has reached the opposite side of the epiphysis E with respect to the insertion end, it can be envisaged to cut away the excess portion of the secondary element with a special cutting instrument.

The secondary elements 34 thus also take advantage of the support on the cortical bone, transferring loads from the damaged portion D of the subchondral bone to the healthy subchondral bone and the healthy cortical portion.

A screw thread 42 is preferably included at the end 40 of the secondary elements 34, corresponding to the end of insertion thereof in the bone during the assembly method described below. The presence of the screw thread 42 allows the secondary elements 34 to be anchored to the cortical bone portion C once it is reached by the end 40 during insertion. Such an anchorage blocks possible sliding of the secondary elements 34 along their respective secondary extension directions Y.

Of course, a further screw thread (not shown) can be included at the other end 38 of the secondary elements 34, so as to anchor the secondary elements 34 more tightly to both opposite cortical portions C.

The first secondary directions Ya, Yb, Yc are preferably parallel to the primary extension direction X of the primary element 12 (see for example FIG. 4). Consequently, the second secondary directions Yd, Ye, Yf are preferably orthogonal to the primary extension direction X of the primary element 12.

More specifically, in the preferred embodiment of FIG. 1, the second secondary directions Yd, Ye, Yf intersect the primary element 12. In other words, the second secondary elements 34d, 34e, 34f pierce the tubular body 14 of the primary element 12, extending through pairs of openings 22a, 22b (only one pair shown in FIG. 1) arranged aligned along the respective second secondary extension direction Yd, Ye, Yf.

The primary element 12 thereby provides further support points for the second secondary elements 34d, 34e, 34f, in addition to the aforementioned support of the relative ends 38, 40 at the cortical bone portions C. The interlacing thus created between the second secondary elements 34d, 34e, 34f and the primary element 12 considerably stabilises the lattice structure 10.

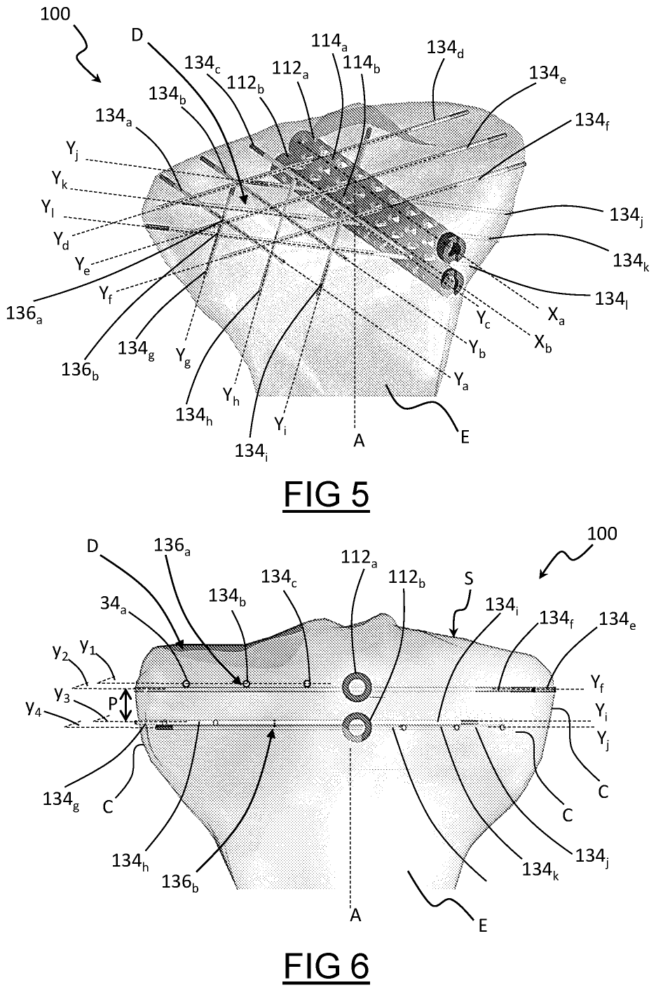

With reference to FIG. 5-6 a lattice structure 100 is now described according to a second embodiment of the invention.

Elements which are identical or similar to the previous embodiment will be indicated below with numerical references increased by 100 each time.

The lattice structure 100 is further reinforced with respect to the lattice structure 10 of the previous embodiment. In fact, it comprises a first primary element 112a and a second primary element 112b, extending along respective primary extension directions Xa, Xb, both antero-posterior directions. For the sake of simplicity, the two primary elements as a whole will sometimes be referred to in the following as 112, as well as the relative primary extension directions as a whole as X.

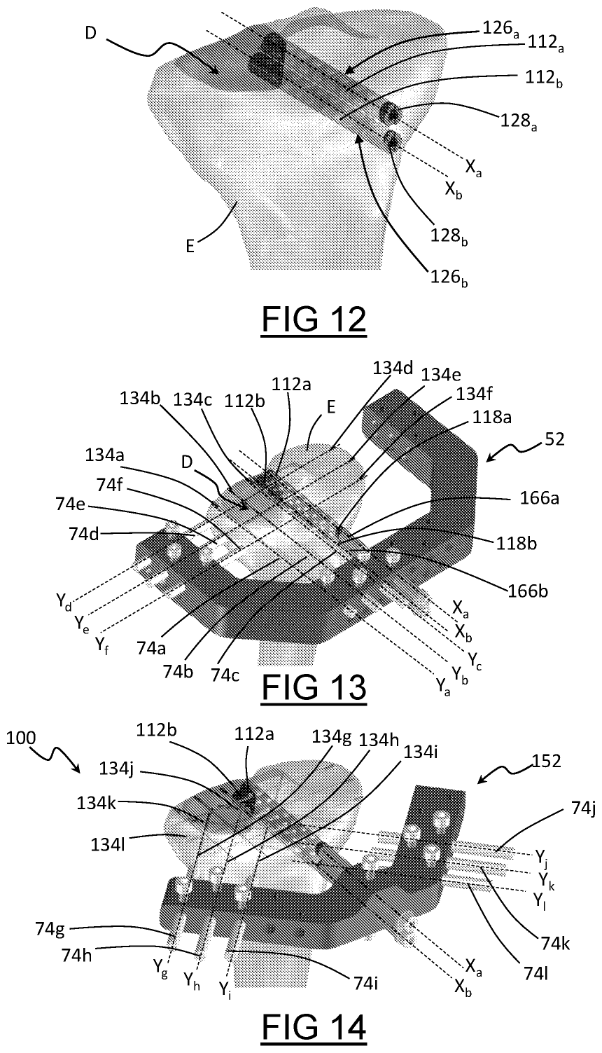

In particular, the secondary extension directions Xa, Xb of the first primary element 112a and the second primary element 112b are aligned with one another in a plane substantially parallel to the longitudinal axis A of the bone of which the bone epiphysis E is part. In addition to offering advantages in terms of increased structural robustness of the lattice structure 100, the inclusion of two primary elements arranged in parallel allows for greater precision in the assembly of the lattice structure 100, as will become clear from the description of the assembly method below with reference to FIG. 12-14.

The lattice structure 100 comprises first secondary elements 134a, 134b, 134c, extending along respective first secondary directions Ya, Yb, Yc, and second secondary elements 134d, 134e, 134f, extending along respective second secondary directions Yd, Ye, Yf. For the sake of simplicity, the secondary elements as a whole will sometimes be referred to in the following as 134, as well as the relative secondary extension directions as a whole as Y.

As in the preceding embodiment, the first secondary directions Ya, Yb, Yc, parallel to one another, lie in the first plane y1 preferably orthogonal to the longitudinal axis A of the bone of which the epiphysis E is part, and the second secondary directions Yd, Ye, Yf, parallel to one another, lie in the second plane y2 also preferably orthogonal to the longitudinal axis A of the bone. The first and second planes y1, y2 are parallel to one another.

Furthermore, the first secondary directions Ya, Yb, Yc are oblique, more in particular orthogonal, with respect to the second secondary directions Yd, Ye, Yf, the first secondary directions Ya, Yb, Yc and the second secondary directions Yd, Ye, Yf intersecting at first intersection points 136a (only one indicated for simplicity in FIG. 5-6).

The second plane y2 extends below the first plane y1, slightly farther away from the joint surface S (see for example FIG. 6) with respect to the first plane y1.

As better seen in FIG. 6, the first and second planes y1 and y2 are close to one another, so that the first secondary elements 134a, 134b, 134c and the second secondary elements 134d, 134e, 134f are substantially side by side or in some cases in contact at the first intersection points 136a.

In the case illustrated in FIG. 5, the lattice structure further comprises third secondary elements 134g, 134h, 134i, extending along respective third secondary directions Yg, Yh, Yi, and fourth secondary elements 134j, 134k, 1341, extending along respective fourth secondary directions Yj, Yk, Yl.

The third secondary directions Yg, Yh, Yi, parallel to one another, extend within a same third plane y3, preferably orthogonal to the longitudinal axis A of the bone of which the epiphysis E is part. Therefore, the third plane y3 is essentially parallel to the first and second planes y1, y2.

Similarly, the fourth secondary directions Yj, Yk, Yl, parallel to one another, extend in the same fourth plane y4, which is also preferably orthogonal to the longitudinal axis A, and therefore substantially parallel to the first plane y1, the second plane y2 and the third plane y3.

Furthermore, the third secondary directions Yg, Yh, Yi are oblique and more in particular orthogonal to the fourth secondary directions Yj, Yk, Yl, the third secondary directions Yg, Yh, Yi and the fourth secondary directions Yj, Yk, Yl intersecting at second intersection points 136b (only one indicated for simplicity in FIG. 5-6).

The fourth plane y4 extends below the third plane y3, slightly farther away from the joint surface S (see for example FIG. 6) with respect to the first plane y1.

The third and fourth planes y3 and y4 are close to one another, so that the third secondary elements 134g, 134h, 134i and the fourth secondary elements 134j, 134k, 1341 are substantially side by side or in some cases in contact at the second crossing points 136b.

Furthermore, the third plane y3 extends below the second plane y2, spaced from the latter by a distance P, farther away from the joint surface S. The distance P is preferably of the order of magnitude of the diameter of the primary elements 112. Therefore, two lattice substructures are distinguished in the lattice structure 100, a first lattice substructure formed by the juxtaposition of the first and second secondary elements 134a, 134b, 134c; 134d, 134e, 134f, and a second lattice substructure formed by the juxtaposition of the third and fourth secondary elements 134g, 134h, 134i; 134j, 134k, 1341, said lattice substructures being spaced apart by a distance P.

Furthermore, the third secondary directions Yg, Yh, Yi are oblique with respect to the first secondary directions Ya, Yb, Yc and the second secondary directions Yd, Ye, Yf. Similarly, the fourth secondary directions Yj, Yk, Yl are also oblique with respect to the first secondary directions Ya, Yb, Yc and the second secondary directions Yd, Ye, Yf.

More specifically, the third secondary directions Yg, Yh, Yi are oriented at about 45° with respect to the first secondary directions Ya, Yb, Yc and the second secondary directions Yd, Ye, Yf. Consequently, given the orthogonality between first and second secondary directions Ya, Yb, Yc; Yd, Ye, Yf and between third and fourth secondary directions Yg, Yh, Yi; Yj, Yk, Yl, the fourth secondary directions Yj, Yk, Yl are also oriented at about 45° with respect to the first secondary directions Ya, Yb, Yc and the second secondary directions Yd, Ye, Yf.

In this embodiment, the second secondary directions Yd, Ye, Yf intersect the first primary element 112a and are in particular orthogonal to the primary extension direction Xa of the primary element 112a. Therefore, the second secondary elements 134d, 134e, 134f pierce the tubular body 114a of the first primary element 112a, extending through pairs of openings arranged aligned along the respective second secondary extension direction Yd, Ye, Yf. The third and fourth secondary directions Yg, Yh, Yi; Yj, Yk, Yl intersect the second primary element 112b, and are oriented at about 45° with respect to the first and second primary extension directions Xa, Xb of the primary elements 112. Therefore, the third and fourth secondary elements 134g, 134h, 134i; 134j, 134k, 1341 pierce the tubular body 114b of the second primary element 112b, extending through pairs of openings arranged in alignment along the respective third and fourth secondary directions Yg, Yh, Yi; Yj, Yk, Yl.

In this configuration, each primary element 112a, 112b offers further support points to the first and second lattice substructures mentioned above, respectively.

With respect to the previously described lattice structure 10, the lattice structure 100 provides an even denser lattice of secondary elements 134 in the degenerated bone portion D, determined by the presence of a greater number of intersection points 136 between secondary elements 134 which provides even greater stability to the structure 100 as a whole.

The primary elements 112 and the secondary elements 134 of this embodiment reach and cross opposite portions of the cortical bone C, so as to have respective partially exposed ends of said opposite portions of the cortical bone C, in a manner entirely analogous to that described for the embodiment of FIG. 1-4.

FIG. 7 illustrates a lattice structure 200 in accordance with a third embodiment of the invention.

The lattice structure 200 comprises a first primary element 212a extending along a primary antero-posterior extension direction Xa. The lattice structure 200 further comprises a second primary element 212b and a third primary element 212c, extending along a second primary extension direction Xb and a third primary extension direction Xc, respectively. The first, second and third primary extension directions Xa, Xb, Xc lie substantially in a same first plane y1. For the sake of simplicity, the three primary elements as a whole will sometimes be referred to in the following as 212, as well as the relative primary extension directions as a whole as X.

As shown, the second and third primary elements 212b, 212c have a third diameter d₃ which is smaller with respect to the first diameter d₁ of the first primary element 212a, and in particular comparable with the diameter of the openings obtained in the tubular body 214a of the first primary element 212a. Thereby, the second and third primary elements 212b, 212c pierce the tubular body 214a of the first primary element 212a, extending through pairs of openings 222a, 222b and 222c, 222d, arranged aligned along the second and third primary extension directions Xb, Xc, respectively.

The lattice structure 200 further comprises three first secondary elements 234a, 234b, 234c extending along respective first secondary directions Ya, Yb, Yc, and two second secondary elements 234d, 234e, extending along respective second secondary directions Yd, Ye. For the sake of simplicity, the secondary elements as a whole will sometimes be referred to in the following as 234, as well as the relative secondary extension directions as a whole as Y.

As described for the previous embodiments, the first secondary directions Ya, Yb, Yc are parallel and coplanar to one another in the first plane y1, preferably orthogonal to the longitudinal axis of the bone of which the epiphysis E is part, and the second secondary directions Yd, Ye are parallel and coplanar to one another in the second plane y2, preferably orthogonal to the longitudinal axis A of the bone of which the epiphysis E is part. The second plane y2 is parallel to the first plane y1 and close thereto, as described for the previous embodiments. The first secondary directions Ya, Yb, Yc are oblique, in particular orthogonal, with respect to the second secondary directions Yd, Ye, defining intersection points 236 (only one shown in FIG. 7).

The first secondary directions Ya, Yb, Yc are parallel to the first primary extension direction Xa of the first primary element 212a, and orthogonal to the second and third primary extension directions Xb, Xc of the second and third primary elements 212b, 212c. The second secondary directions Yd, Ye are instead parallel to the second and third primary extension directions Xb, Xc of the second and third primary elements 212b, 212c, and orthogonal to the first primary extension direction Xa of the first primary element 212a.

In this embodiment, the second secondary directions Yd, Ye intersect the primary element 212a, and the first secondary directions Ya, Yb, Yc intersect the second and third primary elements 212b, 212c.

As in all the previous embodiments, the primary elements 212 and secondary elements 234 of this embodiment reach and cross opposite portions of cortical bone C, so as to have respective partially exposed ends of said opposite portions of cortical bone C.

The assembly of the primary elements 212a, 212b, 212c of the lattice structure 200 forms a true reinforcing frame supported on the cortical portions C, particularly stable and capable of providing multiple additional support points for the secondary elements 234, at the points where the latter pierce the primary elements 212.

With reference to FIG. 8-11, a kit of parts 50 for assembling a lattice structure 10, 100, 200 according to any of the above-described embodiments is now described.

The kit of parts 50 comprises one or more primary elements 12 and a plurality of secondary elements 34, as illustrated in FIG. 8-9 already described above. In particular, the number and size of the primary and secondary elements 34 is determined based on a patient-specific analysis to determine the load situation of the degenerated joint, in order to design a lattice structure tailored to the needs of the patient.

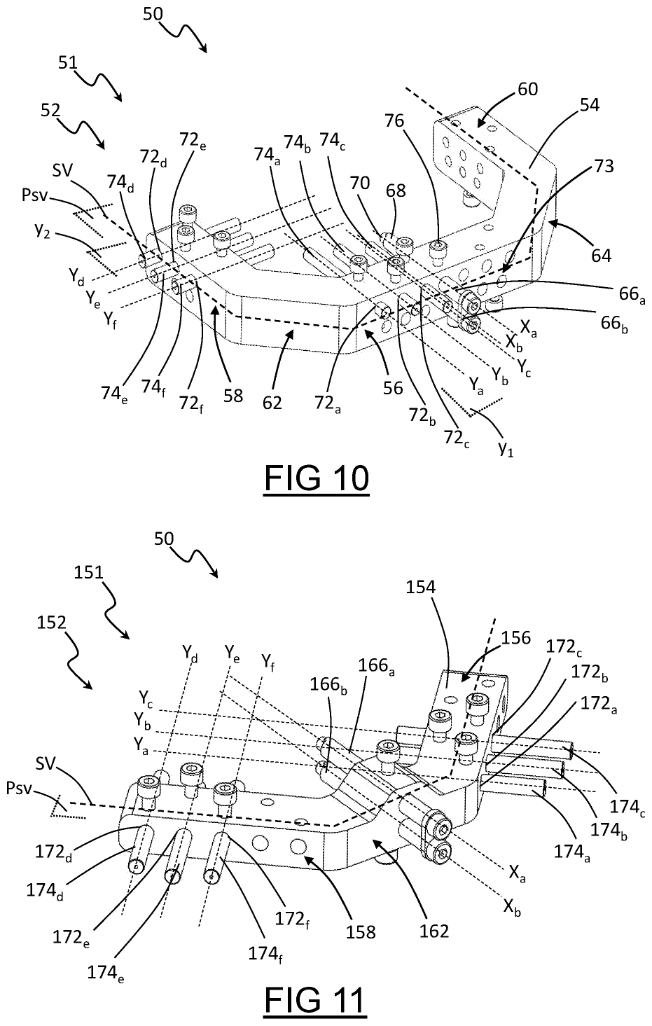

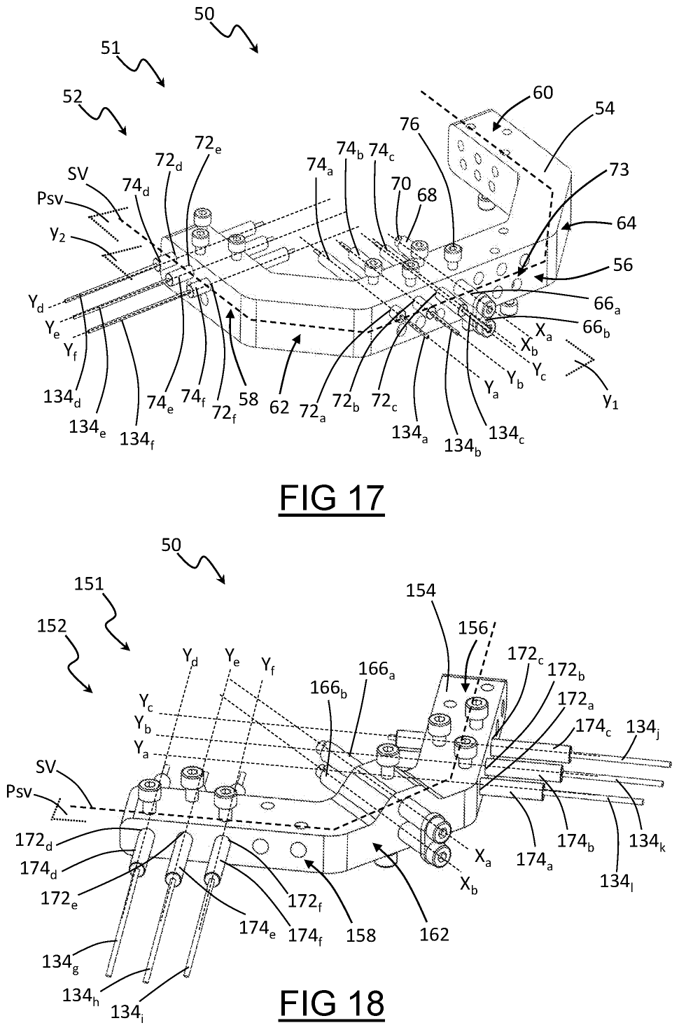

The kit of parts 50 according to the invention further comprises a template assembly 51, illustrated schematically in FIGS. 10 and 17, configured to facilitate the assembly of the lattice structure 10, 100, 200 according to the invention.

The template assembly 51 includes a surgical template 52 having a generically elongated main body 54 comprising a first rectilinear portion 56 and a second rectilinear portion 58 extending substantially orthogonal to the first rectilinear portion 56. The template 52 preferably also comprises a third rectilinear portion 60 extending substantially orthogonal to the first rectilinear portion 56 and substantially parallel to the second rectilinear portion 58.

A development direction SV of the main body 54 of the template 52, indicated by a broken line in FIG. 10, is contained in a development plane Psv. The main body 54 of the template 52 is therefore substantially planar, disregarding its thickness in the direction transversal to the development plane Psv.

The first rectilinear portion 56 extends between the second rectilinear portion 58 and the third rectilinear portion 60, the second and third rectilinear portions 58, 60 being at free ends of the main body 54 of the template 52. The second and third rectilinear portions 58, 60 extend on the same side as the first rectilinear portion 56, resulting in a substantially C-shaped conformation of the main body 54 of the template 52, adapted to surround the bone epiphysis E on three sides to assemble the lattice structure 10, 100, 200.

The main body 54 of the template 52 further comprises rectilinear connecting portions 62, 64 extending between the first rectilinear portion 56 and the second rectilinear portion 58, and between the first rectilinear portion 56 and the third rectilinear portion 60, respectively. In particular, the connecting portions 62 are oriented at about 45° with respect to the first, second and third rectilinear portions 56, 58, 60.

The template 52 further comprises a first pin 66a fixed within a corresponding first through hole (not shown), obtained in the main body 54 of the template 52, in particular in the first rectilinear portion 56, and extending substantially parallel to the second and third rectilinear portions 58, 60 of the main body 54 of the template 52.

The first pin 66a bears, at a proximal end thereof 68, a first fixing member 70 adapted to cooperate with the fixing seat 32 made at the end 18 of a primary element 12, illustrated in FIG. 8. The first fixing member 70 is, for example, a threaded element adapted to cooperate with the screw thread included at the end 18 of the primary element 12 of FIG. 8.

Preferably, the template 52 also comprises a second pin 66b, as illustrated in FIG. 10, fixed within a corresponding second through hole (not indicated), obtained in the main body 54 of the template 52, again in the first rectilinear portion 56, parallel, in a plane transversal to the first rectilinear portion 56, to the first through hole housing the first pin 66a. The second pin 66b bears, at a proximal end thereof (not indicated), a second fixing member (not indicated) adapted to cooperate with the fixing seat 32 made at the end 18 of a further primary element 12. For example, the first and second pins 66a, 66b of the template 52 could fix to the first and second primary elements 112a, 112b of the lattice structure 100 illustrated in FIG. 5-6.

Advantageously, the main body 54 of the template 52 bears first secondary holes 72a, 72b, 72c, a longitudinal axis of which defines, during the assembly of the lattice structure 10, 100, 200, the first secondary extension directions Ya, Yb, Yc of the first secondary elements 32a, 32b, 32c in the damaged portion D of subchondral bone, oriented parallel to one another and extending in a same plane y1. The first secondary extension directions Ya, Yb, Yc are also parallel to the first pin 66a and the second pin 66b.

The main body 54 of the template 52 also bears second secondary holes 72d, 72e, 72f, whose longitudinal axis defines, during the assembly of the lattice structure 10, 100, 200, the second secondary extension directions Yd, Ye, Yf of the second secondary elements 32d, 32e, 32f in the damaged portion D of subchondral bone, oriented parallel to one another and extending in a same plane y2 parallel and close to the first plane y1.

Preferably, the template 52 further comprises first guides 74a, 74b, 74c and second guides 74d, 74e, 74f inserted in the first and second secondary holes 72a, 72b, 72c; 72d, 72e, 72f, respectively, made in the form of tubular elements designed to facilitate the correct alignment of the secondary elements 34, 134, 234 in the bone epiphysis E. Adjustment screws 76 (only one indicated in FIG. 10) can further be included for adjusting the position and/or the fixing of the first and second guides 74a, 74b, 74c; 74d, 74e, 74f.

The template 52 can comprise, as shown, further groups of holes indicated overall by reference 73, which define possible additional extension directions for further secondary elements, based on the envisaged configuration of the lattice structure to be assembled.

The template assembly 51 can include secondary elements 134 pre-assembled to the template 52, as schematically illustrated in FIG. 17.

In the illustrated embodiment, the template assembly 51 comprises first secondary elements 134a, 134b, 134c inserted into the first secondary holes 72a, 72b, 72c of the template 52, and second secondary elements 134d, 134e, 134f inserted into the second secondary holes 72d, 72e, 72f.

More in particular, the first secondary elements 134a, 134b, 134c are inserted into the first guides 74a, 74b, 74c in turn inserted into the first secondary holes 72a, 72b, 72c of the template 52, and the second secondary elements 134d, 134e, 134f are inserted into the second guides 74d, 74e, 74f in turn inserted into the second secondary holes 72d, 72e, 72f.

In particular, the first and second secondary elements 134a, 134b, 134c; 134d, 134e, 134f are set back in the holes, i.e., protruding more from the distal side of the template 52 and only partially emerging from the proximal side of the template 52. Thereby, during the surgical procedure, it is sufficient to bring the pre-assembled template assembly 51 closer to the bone epiphysis E and introduce the first and second secondary elements 134a, 134b, 134c; 134d, 134e, 134f therein, sliding them in the proximal direction. The first and second secondary elements 134a, 134b, 134c; 134d, 134e, 134f can for example already be made of the correct length so that once inserted they reach and cross opposite portions of cortical bone C of the bone epiphysis E.

The provision of secondary elements 134 pre-assembled on the template 52 speeds up the surgical procedure and considerably reduces the risk of errors in the assembly of the structure, as it advantageously allows to avoid assembly operations which are sometimes quite complex during the surgical procedure.

FIGS. 11 and 18 show a second variant of a template assembly 151, which can be used in combination with or as an alternative to the template assembly 51 including the template 52 of FIGS. 10 and 17 to assemble a lattice structure according to the invention, for example the lattice structure 100 when the template 152 is used in combination with the template 52.

The template assembly 151 includes a surgical template 152 having a main body 154 comprising a first rectilinear portion 156 and a second rectilinear portion 158 extending substantially orthogonal with respect to the first rectilinear portion 156, connected by a rectilinear connecting portion 162 oriented at about 45° with respect to the first and second rectilinear portions 156, 158.

Similarly to that set out for the template 52 according to the previous embodiment, a development direction SV of the main body 154 of the template 152, indicated by a broken line in FIG. 11, is contained in a development plane Psv. The main body 154 of the template 152 is therefore substantially planar, disregarding its thickness in the direction transversal to the development plane Psv.

The first rectilinear portion bears third secondary holes 172g, 172h, 172i with associated third guides 174g, 174h, 174i, defining third secondary extension directions Yg, Yh, Yi, while the second rectilinear portion 158 bears fourth secondary holes 172j, 172k, 1721 with associated fourth guides 174j, 174k, 1741, defining fourth secondary extension directions Yj, Yk, Yl.

Unlike template 52 of FIG. 10, in template 152 the first pin 166a and the second pin 166b, bearing the respective fixing member 170, are fixed to the connecting portion 162 of the main body 154 of the template 152, and are thus oriented at about 45° with respect to the third secondary holes and the fourth secondary holes 172g, 172h, 172i; 172j, 172k, 172l.

For example, the template 152 is configured for fixing the third secondary elements 134g, 134h, 134i and/or fourth secondary elements 134j, 134k, 1341 in the lattice structure 100 illustrated in FIG. 5-6, extending along the third and fourth secondary extension directions Yg, Yh, Yi; Yj, Yk, Yl, oriented at about 45° with respect to the first and second main extension directions Xa, Xb.

It is possible to envisage a template which includes both the features of the template 52 and those of the template 152, for example by including holes in the template 52 on the connecting portion 62 configured to additionally house, if necessary, the pins 166a, 166b.

The template assembly 151 can include secondary elements 134 pre-assembled to the template 152, as schematically illustrated in FIG. 18.

The template assembly 151 comprises first secondary elements 134a, 134b, 134c inserted into the first secondary holes 172a, 172b, 172c of the template 152, and second secondary elements 134d, 134e, 134f inserted into the second secondary holes 172d, 172e, 172f.

More in particular, the first secondary elements 134a, 134b, 134c are inserted into the first guides 174a, 174b, 174c in turn inserted into the first secondary holes 172a, 172b, 172c of the template 152, and the second secondary elements 134d, 134e, 134f are inserted into the second guides 174d, 174e, 174f in turn inserted into the second secondary holes 172d, 172e, 172f.

In particular, the first and second secondary elements 134a, 134b, 134c; 134d, 134e, 134f are set back in the holes, i.e., protruding more from the distal side of the template 152 and only partially emerging from the proximal side of the template 152. Thereby, during the surgical procedure, it is sufficient to bring the pre-assembled template assembly 151 closer to the bone epiphysis E and introduce the first and second secondary elements 134a, 134b, 134c; 134d, 134e, 134f therein, sliding them in the proximal direction. The first and second secondary elements 134a, 134b, 134c; 134d, 134e, 134f can for example already be made of the correct length so that once inserted they reach and cross opposite portions of cortical bone C of the bone epiphysis E.

The provision of secondary elements 134 pre-assembled on the template 152 speeds up the surgical procedure and considerably reduces the risk of errors in the assembly of the structure, as it advantageously allows to avoid assembly operations which are sometimes quite complex during the surgical procedure.

With reference to FIG. 12-14, a method for assembling a lattice structure according to the invention is now described.

In the following, special reference will be made to the assembly of the lattice structure 100 of FIG. 5-6, but it is understood that what will be described also applies, mutatis mutandis, to the other embodiments of the lattice structure disclosed in the present document.

Once the configuration of the lattice structure to be assembled in the bone epiphysis E has been established according to the specific requirements of the patient, and the relative kit of parts 50 comprising one or more primary elements 112, a plurality of secondary elements 134 and the template assemblies 51, 151 respectively including the surgical templates 52 and 152 have been prepared, two housing seats 126a, 126b are prepared by bone coring through the degenerated bone portion D, for the two primary elements 112a, 112b. The housing seats 126a, 126b are through seats and cross through the bone epiphysis E from side to side, thus comprising respective accesses 128a, 130a; 120b, 130b facing opposite portions of cortical bone C of the epiphysis E. The housing seats 126a, 126b are made aligned with each other in a plane substantially parallel to the longitudinal axis A of the bone of which the bone epiphysis E is part.

The first primary element 112a and the second primary element 112b are then inserted into the respective housing seat 126a, 126b (FIG. 12).

The template 52 of the template assembly 51 is then constrained to the first and second primary elements 112a, 112b by screwing the respective fixing members (not visible here) of the first pin 66a and the second pin 66b into the corresponding fixing seats (not visible here) borne by the respective ends 118a, 118b of the first and second primary elements 112a, 112b. The fixing of the template 52 on two pins 66a, 66b allows for greater stability thereof and prevents it from rotating during the subsequent insertion of the secondary elements 134.

The first and second secondary elements 134a, 134b, 134c; 134d, 134e, 134f are then introduced—if not pre-assembled to the template 52 as in the configuration illustrated in FIG. 17—respectively in the first and second guides 74a, 74b, 74c; 74d, 74e, 74f housed in the respective first and second holes (not indicated here) of the template 52, which then automatically impart the first secondary directions Ya, Yb, Yc to the first secondary elements 134a, 134b, 134c, and the second secondary directions Yd, Ye, Yf to the second secondary elements 134d, 134e, 134f.

Once introduced into the first and second guides 74a, 74b, 74c; 74d, 74e, 74f, the first and second secondary elements 134a, 134b, 134c; 134d, 134e, 134f are inserted into the degenerated portion D of subchondral bone by means of a drilling tool, e.g., a drill. The first and second secondary elements 134a, 134b, 134c; 134d, 134e, 134f are pushed into the bone until they reach the cortical portion located on the side of the epiphysis E opposite the insertion side (FIG. 13). With this insertion, the first lattice substructure is assembled, formed by the first and second secondary elements 134a, 134b, 134c; 134d, 134e, 134f arranged orthogonally with respect to one another, with the second secondary elements 134d, 134e, 134f intersecting the first primary element 112a.

Upon completion of this insertion, the template assembly 51 is then removed, releasing the first pin and the second pin 66a, 66b of the template 52 from the ends 118a, 118b of the first and second primary elements 112a, 112b.

The template assembly 151 of the second type is then constrained to the first and second primary elements 112a, 112b by screwing the respective fixing members 170a, 170b of the first pin 166a and of the second pin 166b of the template 152 into the corresponding fixing seats 32a, 32b borne by the ends 118a, 118b of the first and second primary elements 112a, 112b.

The third and fourth secondary elements 134g, 134h, 134i; 134j, 134k, 1341 are then introduced—if not pre-assembled to the template 152 as in the configuration illustrated in FIG. 18—in the third and fourth guides 174g, 174h, 174i; 174j, 174k, 1741 housed in the respective third and fourth holes (not indicated here), which therefore automatically impart third secondary directions Yg, Yh, Yi to the third secondary elements 134g, 134h, 134i, and the fourth secondary directions Yj, Yk, Yl to the fourth secondary elements 134j, 134k, 134l.

Once introduced into the first and second guides 174g, 174h, 174i; 174j, 174k, 1741, the third and fourth secondary elements 134g, 134h, 134i; 134j, 134k, 1341 are inserted into the degenerated portion D of subchondral bone using the drilling tool already used in the previous step. The third and fourth secondary elements 134g, 134h, 134i; 134j, 134k, 1341 are pushed into the bone until they reach the cortical portion located on the side of the epiphysis E opposite the insertion side (FIG. 14). With this insertion, the second lattice substructure is assembled, formed by the third and fourth secondary elements 134g, 134h, 134i; 134j, 134k, 1341 arranged orthogonally with respect to one another, with the third and fourth secondary elements 134g, 134h, 134i; 134j, 134k, 1341 intersecting the second primary element 112b.

At the end of this insertion the lattice structure 100 is fully assembled. The template assembly 151 is then also removed, releasing the first pin and the second pin 166a, 166b of the template 152 from the ends 118a, 118b of the first and second primary elements 112a, 112b.

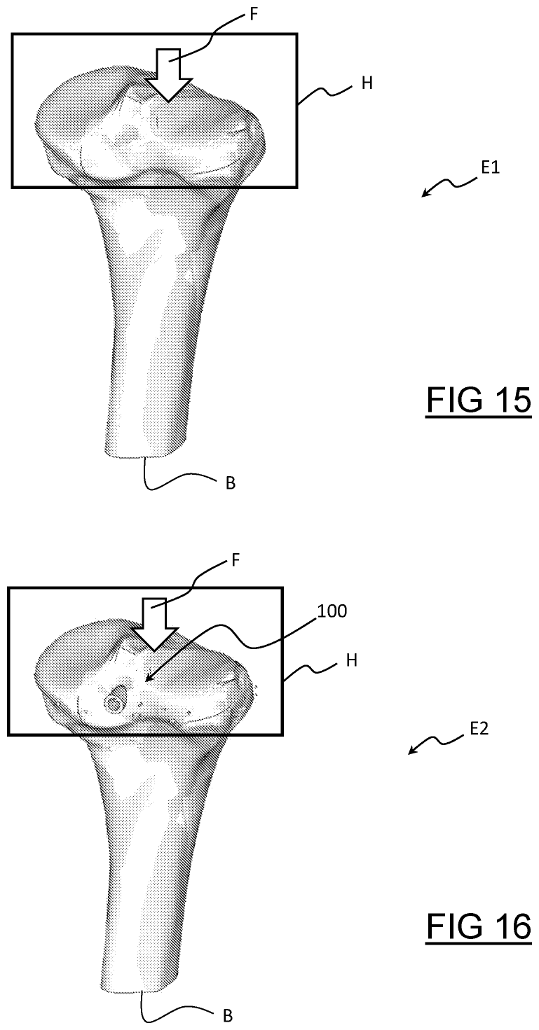

The Applicant has carried out experiments in order to verify the performance of the lattice structure according to the invention, in terms of reducing the load peaks acting on the damaged portion of the bone epiphysis, and reducing the deformations to which such a damaged portion is subjected. Some results of such experiments, which are intended to be illustrative and not limiting of the present invention, are described below with reference to FIG. 15-16.

From diagnostic MRI images of a patient, two three-dimensional models of tibia portions were constructed in the CAD environment, a first model E1 (FIG. 15) without a lattice structure, and a second model E2 (FIG. 16) having a lattice structure analogous to the structure 100 of FIG. that illustrated in FIG. 5, but with a single primary element 112, partially visible in FIG. 16. Both models E1, E2 comprise the head region T and a portion of the distal region of the tibia, extending between the head T and an end B. In the model E2 of FIG. 16 secondary elements with a diameter of 1 mm and a primary element with an external diameter of 6 mm were considered.

The two models E1 and E2 were analysed and compared by means of finite element simulations (FEM) using the FEM module of Creo Elements software (PTC Inc.), imposing an interlocking constraint at the end B of the tibia portion.

In both models E1, E2 a load F of 3200 N acting vertically on the head region H of the tibia was imposed, which is equal to the load which a man of about 75 kg exerts on the tibia during a walk. The distribution of the local equivalent stress values according to Von Mises (MPa) was extracted from the simulation, as well as the distribution of the local deformations on the surface of the head region T.

From the distribution of the equivalent stress according to Von Mises, the peak values of such equivalent stress, acting in the head region H of the tibia, were determined. In model E1, the equivalent stress peaks exceeded 100 MPa, while in model E2, with the lattice structure assembled, the equivalent stress peaks were kept within 70 MPa.

From the distribution of the local deformations of the bone tissue, an overall deformation of the head region H of approximately 1 mm was obtained in model E1, which decreased to approximately 0.77 mm of overall deformation of the head region H in model E2 with the lattice structure assembled.

The above data made it possible to verify that the lattice structure according to the invention allows to decrease by at least 30% the equivalent tension peaks acting in the head region of the tibia where it is assembled, when the tibia is loaded. Similarly, it was verified that the lattice structure is able to decrease bone tissue deformations in the head region of the tibia bearing the structure by more than 20%.

Obviously, a person skilled in the art, in order to satisfy specific and contingent needs, can make numerous modifications and variations to the invention described above while remaining within the scope of protection defined by the following claims.

1. A template assembly comprising
a surgical template including an elongated main body
at least one fixing member configured to cooperate with at least one corresponding fixing seat (32) of at least one rod-shaped and substantially rectilinear rigid primary element, wherein
i) said main body comprises first and second secondary through holes configured to respectively house thread-like and substantially rectilinear first secondary elements and second secondary elements wherein the first secondary holes define respective first secondary extension directions for the first secondary elements, and the second secondary holes define respective second secondary extension directions for the second secondary elements, the first and the second secondary extension directions being oblique to one another, or
ii) said main body comprises third and fourth secondary through holes configured to respectively house thread-like and substantially rectilinear third secondary elements and fourth secondary elements, wherein the third secondary holes define respective third secondary extension directions for the third secondary elements, and the fourth secondary holes define respective fourth secondary extension directions for the fourth secondary elements, the third and fourth secondary extension directions being oblique to one another.

2. The template assembly according to claim 1, wherein:
i) the first secondary extension directions are substantially parallel to one another and lie in a first plane, and the second secondary extension directions are substantially parallel to one another and lie in a second plane, said second plane being substantially parallel to said first plane, or
ii) the third secondary extension directions are substantially parallel to one another and lie in a third plane, and the fourth secondary extension directions are substantially parallel to one another and lie in a fourth plane, said fourth plane being substantially parallel to said third plane.

3. The template assembly according to claim 1, wherein the main body (54, 154) of the template (52, 152) comprises a central portion extending between two free ends, said free ends extending from the same side of the template (52, 152) with respect to said central portion.

4. The template assembly according to claim 2, wherein a development direction of the main body of the template is contained in a development plane.

5. The template assembly according to claim 4, wherein said plane selected from the group consisting of said first plane, said second plane, said third plane, and said fourth plane are substantially parallel to said development plane of the main body of the template.

6. The template assembly according to claim 1, wherein the first secondary holes or the third secondary holes are borne by a first rectilinear portion of the template, and the second secondary holes or the fourth secondary holes are borne by a second rectilinear portion extending substantially orthogonal to said first rectilinear portion (56, 156) of the template,
the first secondary holes or the third secondary holes extending transversally with respect to said first rectilinear portion of the template, and
the second secondary holes or the fourth secondary holes extending transversally with respect to said second rectilinear portion of the template.

7. The template assembly according to claim 6, wherein said first rectilinear portion is defined at the central portion of the main body of the template, and said second rectilinear portion is defined at one of the free ends of the main body of the template.

8. The template assembly according to claim 1 wherein said first rectilinear portion and said second rectilinear portion are respectively defined at the free ends of the main body of the template.

9. The template assembly according to claim 1, wherein the template comprises at least one pin bearing, at one of its proximal ends, said at least one fixing member, said at least one pin extending along a primary extension direction parallel to said development plane of the main body of the template.

10. The template assembly according to claim 9, wherein said at least one pin is borne by the first rectilinear portion of the main body of the template, and is parallel to the first secondary extension directions.

11. The template assembly according to claim 9, wherein said at least one pin is borne by a connecting portion extending between the first rectilinear portion and the second rectilinear portion of the main body of the template and is oriented at about 45° with respect to the third secondary extension directions and the fourth secondary extension directions.

12. The template assembly according to claim 1, wherein the template comprises a first fixing member, configured to cooperate with a corresponding first fixing seat of a rod-shaped and substantially rectilinear rigid first primary element, and a second fixing member, configured to cooperate with a corresponding second fixing seat of a rod-shaped and substantially rectilinear rigid second primary element.

13. The template assembly according to claim 12, wherein the template comprises a first pin bearing said first fixing member at one of its proximal ends, said first pin being substantially parallel to said development plane of the main body of the template, and a second pin bearing said second fixing member at one of its proximal ends, said second pin being substantially parallel to said development plane of the main body of the template and substantially parallel to said first pin.

14. The template assembly according to claim 1, further comprising said thread-like and substantially rectilinear first secondary elements inserted in the first secondary holes, or further comprising thread-like and substantially rectilinear third secondary elements inserted in the third secondary holes.

15. The template assembly according to claim 1, further comprising thread-like and substantially rectilinear second secondary elements inserted in the second secondary holes, or further comprising thread-like and substantially rectilinear fourth secondary elements inserted in the fourth secondary holes.

16. A kit of parts for assembling a lattice support structure for one or more degenerated portions of subchondral bone of a bone epiphysis part of a human or animal joint, said kit of parts comprising:
at least one rod-shaped and substantially rectilinear rigid primary element,
a plurality of substantially rectilinear thread-like secondary elements, comprising first secondary elements and second secondary elements, and
a template assembly according to claim 1;
wherein said at least one primary element has a first transversal dimension greater than a second transversal dimension of said secondary elements, and
wherein the at least one primary element and the secondary elements are configured to reach and cross at least partially, at both the respective ends, cortical bone portions of said bone epiphysis.

17. The kit according to claim 16, wherein said plurality of secondary elements further comprises third secondary elements and fourth secondary elements.

18. The kit according to claim 16, wherein the at least one primary element comprises a tubular body with a circular cross section.

19. The kit according to claim 16, wherein the tubular body of the at least one primary element bears a plurality of through openings.

20. The kit according to claim 19, wherein at least some of the secondary elements are configured to extend through corresponding through openings of the tubular body of the at least one primary element.

21. The kit according to claim 16, wherein the at least one primary element includes a first primary element and a second primary element.

22. The kit according to claim 21, wherein the second primary element is configured to extend through corresponding through openings of the tubular body of the first primary element.

23. The kit according to claim 16, wherein the at least one primary element comprises, at an end thereof, a fixing seat for the template, said fixing seat being adapted to cooperate with said fixing member of the template.

24. The kit according to claim 16, wherein an internal lumen of the at least one primary element is configured to receive a filler selected from an autologous bone graft, a homologous bone graft and a synthetic bone graft, said filler being an autologous bone graft.

25. A lattice support structure for one or more degenerated portions of subchondral bone of a bone epiphysis part of a human or animal joint, comprising:
at least one rod-shaped and substantially rectilinear rigid primary element, configured to be housed within said bone epiphysis extending at least partially through said degenerated portion of subchondral bone along a respective primary extension direction, and
a plurality of thread-like substantially rectilinear secondary elements, configured to be housed within said bone epiphysis extending at least partially through said degenerated portion of subchondral bone along respective secondary extension directions, comprising first secondary elements configured to extend along respective first secondary extension directions, and second secondary elements configured to extend along respective second secondary extension directions, the first and the second secondary extension directions being oblique to one another,
wherein said at least one primary element has a first transversal dimension (d1) greater than a second transversal dimension of said secondary elements, and
wherein the at least one primary element and the secondary elements are configured to reach and cross at least partially, at respective opposite ends, cortical bone portions of said bone epiphysis.

26. The lattice support structure according to claim 25, wherein the first secondary extension directions of the first secondary elements lie in a first plane, and/or the second secondary extension directions of the second secondary elements lie in a second plane.

27. The lattice structure according to claim 26, wherein the first plane and the second plane are substantially parallel to one another.

28. The lattice structure according to claim 25, wherein the first secondary extension directions of the first secondary elements are substantially parallel to one another, and/or the second secondary extension directions of the second secondary elements are substantially parallel to one another.

29. The lattice structure according to claim 25, wherein the first secondary extension directions of the first secondary elements are substantially orthogonal to the second secondary extension directions of the second secondary elements.

30. The lattice structure according to claim 25, wherein the plurality of secondary elements further comprises third secondary elements configured to extend along respective third secondary extension directions, and fourth secondary elements configured to extend along respective fourth secondary extension directions, the third and fourth secondary extension directions being oblique to one another.

31. The lattice structure according to claim 30, wherein said third secondary extension directions lie in a third plane, and said fourth secondary extension directions lie in a fourth plane substantially parallel to said third plane,
wherein said third plane and said fourth plane are substantially parallel to said first plane and/or to said second plane.

32. The lattice structure according to claim 30, wherein the third secondary extension directions of the third secondary elements are substantially parallel to one another, and/or the fourth secondary extension directions of the fourth secondary elements (134j, 134k, 134l) are substantially parallel to one another,
wherein the third secondary extension directions are substantially orthogonal to the fourth secondary extension directions, and
wherein the third secondary extension directions are oriented at about 45° with respect to said first secondary extension directions of the first secondary elements and/or to said second secondary extension directions of the second secondary elements.

33. The lattice structure according to claim 25, wherein the primary extension direction of at least one primary element is substantially orthogonal to said second secondary extension directions of the second secondary elements.

34. The lattice structure according to claim 25, wherein the at least one primary element comprises a tubular body (14) with a circular cross section.

35. The lattice structure according to claim 34, wherein the tubular body of the at least one primary element bears a plurality of through openings.

36. The lattice structure according to claim 35, wherein at least some of the secondary elements are configured to extend through corresponding through openings of the tubular body of the at least one primary element.

37. The lattice structure according to claim 25, wherein the at least one primary element includes a first primary element (212a) configured to extend along a first primary extension direction, and a second primary element configured to extend along a second primary extension direction, the first primary extension direction and the second primary extension direction being oblique to one another.

38. A method for assembling a lattice support structure for a degenerated portion of subchondral bone of a bone epiphysis part of a human or animal joint, comprising the steps of:
arranging at least one rod-shaped, substantially rectilinear rigid primary element;
arranging a plurality of substantially rectilinear thread-like secondary elements, comprising first secondary elements and second secondary elements;
in which said at least one primary element has a first transversal dimension greater than a second transversal dimension of said secondary elements;
arranging a surgical template including an elongated main body having at least one fixing member configured to cooperate with at least one corresponding fixing seat of at least one rod-shaped and substantially rectilinear rigid primary element, wherein the main body of the template includes first and second secondary through holes configured to respectively house first secondary elements and second secondary elements which are thread-like and substantially rectilinear, in which the first secondary holes define respective first secondary extension directions for the first secondary elements, and the second secondary holes define respective second secondary extension directions for the second secondary elements, the first and second secondary extension directions being oblique to one another.
forming, by means of bone coring, at least one housing seat through said degenerated bone portion for the at least one primary element, said housing seat crossing the bone epiphysis from side to side and comprising accesses facing opposite cortical bone portions of said bone epiphysis;
inserting the at least one primary element into said at least one housing seat;
constraining the surgical template to said at least one primary element by fixing the at least one fixing member of the template at the at least one corresponding fixing seat of said at least one primary element, accessing said at least one fixing seat through one of the accesses of the housing seat;
inserting the secondary elements into the degenerated portion of subchondral bone using a drilling tool, sliding the secondary elements through the secondary holes of the template; and
removing the template assembly by releasing the at least one fixing member from the corresponding at least one fixing seat of the at least one primary element.

39. The method according to claim 38, further comprising a step of anchoring one or both ends of each secondary element to said cortical bone portion by means of anchoring elements.

40. The method according to claim 38, further including a preliminary step of identifying one or more degenerated subchondral bone portions within the bone epiphysis, wherein said step of identifying said one or more degenerated bone portions includes defining a three-dimensional model of loads acting in a known load situation from diagnostic images of the patient.

41. The method according to claim 39, further including
a step of analysing said three-dimensional model by means of finite element analysis (FEM), and
a step of modelling the components of the lattice structure based on evidence of the FEM analysis.
